(19) United States
(12) Patent Application Publication
(10) Pub. No.: US 2007/0298002 A1
(43) Pub. Date: Dec. 27, 2007

(54) Decorative Cosmetic Product Having a High Water Content

(76) Inventor: Salvatore J. Barone, New York, NY (US)

Correspondence Address: AKERMAN SENTERFITT, P.O. BOX 3188, WEST PALM BEACH, FL, 33402-3188, US

(21) Appl. No.: 11/574,706

(22) PCT Filed: Jul. 27, 2006
(86) PCT No.: PCT/EP06/64736

§ 371 Date: Mar. 5, 2007

(30) Foreign Application Priority Data
Jul. 28, 2005 (DE) 102005036497.7

(51) Int. Cl. A61K 8/18; A61Q 1/06; A61Q 1/08; A61Q 1/10

(52) U.S. Cl. 424/64

(57) ABSTRACT

Decorative cosmetic product having a high water content The invention relates to a decorative cosmetic product having a high water content. The product comprises 70-92% by weight of water, 2.0-30% by weight of pigments, 2-8% by weight of a gel-forming agent with emulsifying properties comprising a mixture of sodium acrylate copolymers, hydrogenated polyisobutene, plant-based phospholipids, a polyglyceryl acylate and an oil, and the remainder up to 100% of said product is made up of cosmetic auxiliaries, carriers, active agents or mixtures thereof. Despite of the cooling effect the product with a stabile cosmetic formula has an extraordinary transfer stability. In the case of lipsticks, a glossy product is obtained with accordingly good transfer stability.

The invention relates to a decorative cosmetic product having a high water content.

Cosmetic preparations such as make-ups, mascaras, rouges, etc., which belong to the decorative preparations, contain in most cases just 10-35% water when they are cream products and mostly less than 70% water when they are fluids, and in the latter case thixotroping agents and surface-active agents are added.

Transfer resistant high lustre lipstick compositions are e.g. known from U.S. Pat. No. 6,036,947 (Barone et al.) which use 10-70% of a volatile solvent i.e. silicones or paraffinic hydrocarbons. Other transfer resistant cosmetic compositions of WO 97/17058 (Drechsler et al.) use silicone compounds such as organosiloxan resins and diorganopolysiloxane polymers.

The object of the invention is to provide decorative cosmetic products which have a high water content, contain no drying solvents and adhere well to the skin while at the same time having a very good transfer stability.

The new products comprise 70-92% by weight of water, 2.0-30% by weight of pigments, 2-8% by weight of a gel-forming agent with emulsifying properties comprising a mixture of sodium acrylate copolymers, hydrogenated polyisobutene, plant-based phospholipids, a polyglyceryl acylate and an oil. The remainder up to 100% of said product is made up of cosmetic auxiliaries, carriers, active agents or mixtures thereof, all percentages being relative to the product's total weight.

Preferred are products which do not utilize “drying” solvents such as silicone fluids such as Dimethicone, Cyclomethicone etc., paraffinic hydrocarbons such as pentane, hexane, heptane or C₈₋₂₀ isoparaffines, or monovalent alcohols such as ethanol, isopropanol etc.

The products preferably contain 73-90% by weight of water, particularly 84-90% by weight. Despite the very high water content, the stability of the cosmetic formulation is ensured and an excellent transfer stability is achieved in addition to the cooling effect. Specifically in the case of lipsticks, a glossy product is obtained whose transfer stability is extraordinarily good.

Advantageously, the products can contain 3-20% by weight of pigments, more preferred 5-20% by weight. A content of 8-15% by weight being particularly preferred. Said pigments are preferably untreated pigments.

The gel-forming agent advantageously makes up 3-7%, especially 2-5% by weight. The gel-forming agent is composed of three components and contains a sodium acrylate copolymer, advantageously a copolymer of sodium acrylate with acryloyldimethyl taurate. The phospholipid contained in the gel-forming agent is advantageously a plant-based phospholipid, e.g. stemming from soya oil, sunflower seed oil, rice oil and mixtures thereof. Preferred phospholipids are phosphatidylcholine, phosphatidylethanolamine, phosphatidylinositol, phosphatidylserine, phosphatidic acid and lysolecithins as well as mixtures thereof.

The polyglyceryl acylate contained in the gel-forming agent is preferably a polyglyceryl stearate, polyglyceryl distearate or polyglyceryl linoleate, particularly a polyglyceryl-10 stearate.

A preferred gel-forming agent is DC Odyssee® Gel (manufactured by Lucas Meyer Cosmetics S.A. Thiais, France) with INCI name Sodium Acrylates Copolymer & Hydrogenated & Sunflower Polyisobutene Phospholipids & Polyglyceryl-10 Stearate % Sunflower Seed Oil.

The inventive product can further contain cosmetic auxiliaries and carriers as they are commonly used in such preparations, e.g. preservatives, colourants, thickeners, fragrances, alcohols, polyols, esters, another gel-forming agent, polar and non-polar oils, polymers, copolymers, surface-active agents, waxes (not preferred), stabilizers.

Additional cosmetic active agents which can be comprised include e.g. inorganic and organic sunscreens, scavengers, moisturizing substances (not preferred), vitamins, enzymes, vegetable active agents, polymers, antioxidants, anti-inflammatory natural active agents, asymmetric lamellar aggregates carrying oxygen according WO 94/000109, disintegration products of yeast or plant substances.

Pigments, pigment mixtures or powders with a pigment-like effect may include, for example, iron oxides, aluminum silicates such as ochre, titanium oxide, mica, kaolin, manganese containing clays such as umber and red bole, calcium carbonate, talc, mica-titanium oxide, mica-titanium oxide-iron oxide, bismuth oxychloride, nylon beads, ceramic beads, expanded and non-expanded synthetic polymer powders, powdery natural organic compounds such as milled solid algae, milled plant parts, encapsulated and non-encapsulated cereal starches.

Further used active agents are antioxidants e.g. vitamins such as vitamin C and derivatives thereof, for example, ascorbic acetate, ascorbic phosphate, and ascorbic palmitate; vitamin A and derivatives thereof; folic acid and derivatives thereof; vitamin E and derivatives thereof, such as tocopheryl acetate; flavones or flavonoids; amino acids, such as histidine, glycine, tyrosine, tryptophan, and derivatives thereof; imidazole such as cis- or trans-urocaninic acid and their derivatives; peptides such as D,L-carnosine, D-carnosine, L-carnosine and their derivatives; carotenoids and carotenes such as, for example, α-carotene, β-carotene; lycopine; uric acid and derivatives thereof; α-hydroxy acids such as citric acid, lactic acid, malic acid; a-hydroxy fatty acids such as palmitic acid, phytic acid, lactoferrin; stilbenes and their derivatives; mannose and their derivatives; liponic acid and their derivatives such as dihydro liponic acid; ferula acid and their derivatives; thiols such as glutathione, cysteine and cystine. It is moreover advantageous to add as active agent water and/or oil soluble UVA or UVB filters or both. Advantageous oil-soluble UVB filters include 4-amino benzoic acid derivatives such as 4-(dimethylamino)-benzoic acid-(2-ethylhexyl) ester; esters of cinnamic acid such as 4-methoxy cinnamic acid (2-ethylhexyl)ester, benzophenone derivatives such as 2-hydroxy-4-methoxy benzophenone; 3-benzylidene camphor derivatives such as 3-benzylidene camphor.

Preferred oil-soluble UV filters are Benzophenone-3, Butyl-Methoxybenzoylmethane, Octyl Methoxycinnamate, Octyl Salicylate, 4-Methylbenzylidene Camphor, Homosalate and Octyl Dimethyl PABA.

Water-soluble UVB filters are, for example, sulfonic acid derivatives of benzophenone or of 3-benzylidene camphor or salts, such as Na or K salts, of 2-phenyl benzimidazole-5-sulfonic acid.

UVA filters include dibenzoyl methane derivatives such as 1-phenyl-4-(4′-isopropanol phenyl) propane-1,3-dione, Butyl Methoxybenzoyl-methane or Menthyl Anthranilate.

Special preferred are Benzophenone-3, Butyl Methoxydibenzoylmethane, Octyl Methoxycinnamate, Octyl Salicylate, 4-Methylbenzylidene Camphor, Homosalate, Octocrylene, Ethylhexyl Methoxycinnamate, Isoamyl-p-Methoxycinnamate, Octyl Dimethyl PABA, Ethylhexyltriazone, Diethylhexyl Butamido Triazone, Ethylhexyl Salicylate, Methylene Bis-Benzotriazolyl Tetramethylbutylphenol, Disodium Phenyl Dibenzimidazole Tetrasulfonate, Bis-Ethylhexyloxyphenol Methoxyphenyl Triazine. Preferred as sunscreen filters are also inorganic pigments on the basis of metal oxides, such as TiO₂, SiO₂, ZnO, Fe₂O₃, ZrO₂, MnO, Al₂O₃, which can also be used in mixtures thereof. Especially preferred as inorganic pigments are agglomerate substrates of TiO₂ and/or ZnO according to WO99/06012 which have a contents of spherical and porous SiO₂ particles, wherein the SiO₂ particles have a particle size in the range of 0.05 μm to 1.5 μm, and, in addition to the SiO₂ particles, other inorganic particle-like substances with spherical structure are present, wherein the spherical SiO₂ particles form defined agglomerates with the other inorganic substances with a particle size in the range of 0.06 mm to 5 μm.

As auxiliaries used waxes (not preferred) may be selected among natural plant waxes, animal waxes, natural and synthetic mineral waxes and synthetic waxes. There are included carnauba wax, candelilla wax, ozokerite, beeswax, montan wax, wool wax, ceresine, micro-waxes, paraffin waxes, petrolatum, silicon wax, polyethylene glycol waxes or polyethylene glycolester waxes. The addition of waxes is not preferred.

As auxiliaries used oils can be usual cosmetic oils such as mineral oil; hydrogenated polyisobutene; squalane from synthetic or natural sources; cosmetic esters or ethers which can be branched or unbranched, saturated or unsaturated; vegatable oils; or mixtures of two or more therof.

Especially suitable oils are, for example mineral oils, hydrogenated polyisobutene, polyisoprene, squalane, tridecyltrimellitate, trimethylpropane triisostearate, isodecylcitrate, neopentyl glycol diheptanoate, PPG-15-stearyl ether, as well as preferred plant oils such as Calendula oil, Jojoba oil, Avocado oil, Macadamia nut oil, Castor oil, Cocoa butter, Coconut oil, Corn oil, Cotton seed oil, Olive oil, Palm kernel oil, Rapeseed oil, Safflower seed oil, Sesame seed oil, Soybean oil, Sunflower seed oil, Wheat germ oil, Grape kernel oil, Kukui nut oil, Thistle oil, and mixtures thereof.

Depending upon the oils selected, the cosmetic properties of the solid composition, such as softness, hardness, spreading effect, are affected.

Polyols, which are also possible auxiliaries for the products of the invention, are e.g. propylene glycol, dipropylene glycol, ethylene glycol, isoprene glycol, glycerin, butylene glycols, sorbitol and mixtures thereof. The share of the polyol is in the range of 0.1 to 10% by weight. Suitable esters or ethers of polyols are, for example, Propylene Glycol Dioctanoate, Propylene Glycol Dicaprylate 2,30 Dicaprate, Tridecyl Stearate/Neopentyl glycol dicaprylate dicaprate/Tridecyl trimellitate, Neopentyl Glycol Dioctanoate, Isopropyl Myristate, Diisopropyl Dimer Dilinoleate, Trimethylpropane Triisostearate, Myristyl Ether, Stearyl Ether, Cetearyl Octanoate, Butyl Ether, Dicaprylyl Ether, Fomblino HC25.

Suitable further cosmetic gel-forming agents include carbomer, xanthan gum, carrageenan, acacia gum, guar gum, silicone gum, agar-agar, alginates and tyloses, carboxymethyl cellulose, hydroxyethyl cellulose, quaternized cellulose, quaternized guar, certain polyacrylates, polyvinyl alcohol, polyvinylpyrrolidone, montmorillonit.

The use of the cosmetic preparations according to the invention for decorative cosmetics can be realized e.g. in the form of makeup, liner, lipstick, eye shadow, concealer, mascara, foundation, rouge, eye-liner, lip-liner etc. The manufacture of such products is carried out in a way known to a person skilled in the art.

A special preferred product is a transfer resistant, high shine lipstick. The lipstick has a water content of 85-90% by weight and is free of waxes, silicones, C₁₋₄ alcohols and/or paraffinic hydrocarbons. Preferred are also other products of decorative cosmetic which are free of waxes, silicones, C₁₋₄ alcohols and/or paraffinic hydrocarbons.

In consumer tests a lipstick according to the invention is applied on lips of test persons. After drying the lips are blotted with a tissue. In all cases no traces of lipstick are found on the tissue. That means a high transfer resistance. Further the test persons are acknowledged the unique high shine and excellent smooth handling.

In the following, the invention shall be described in detail by examples. All percent figures are by weight if not other set out.

### EXAMPLE 1

### Make Up I

| Water | q.s. ad 100 |
|---|---|
| Gelling agent (DC Odessey ®) | 5.0 |
| Preservation | 0.5 |
| Color pigments | 3.2 |
| Talkum | 4.3 |

The pigments and fillers are milled and dispersed in water of 80-83° C. The separate heated (about 80° C.) gelling agent is added to the water phase under stirring. With continued stirring and after cooling preservation agents and fragrances are added, respectively. The stirring is further continued until the product thickens to the wished consistence.

### EXAMPLE 2

### Make Up II

| Water | q.s. ad 100 |
|---|---|
| Preservation | 0.3 |
| EDTA | 0.1 |
| Titanium Dioxide | 11.0 |
| Color pigments | 2.1 |
| Gelling agent (DC Odessey ®) | 6.5 |
| Fragrance | 0.2 |
| Butylene glycol | 6.0 |

### EXAMPLE 3

### Eye Shadow

| Water | q.s. ad 100 |
|---|---|
| Preservation | 0.3 |
| EDTA | 0.1 |
| Gelling agent (DC Odessey ®) | 5.0 |
| Iron Oxide&Mica&Silane | 8.0 |
| Flamenco Twlight Red | 8.0 |
| Color pigments | 0.9 |
| Glycerine (99%) | 0.5 |

### EXAMPLE 4

### Concealer

| Water | q.s. ad 100 |
|---|---|
| Gelling agent (DC Odessey ®) | 3.5 |
| Preservation | 0.3 |
| Color pigments | 3.2 |
| Talc | 4.3 |

### EXAMPLE 5

### Mascara

| Water | q.s. ad 100 |
|---|---|
| Preservations | 0.3 |
| EDTA | 0.1 |
| Color pigments | 13.1 |
| Gelling agent (DC Odessey ®) | 5.0 |
| Butylene glycol | 6.0 |

### EXAMPLE 6

### Lipstick

| Water | q.s. ad 100 |
|---|---|
| Preservations | 0.3 |
| EDTA | 0.1 |
| Mica&Titanium Dioxide&Iron Oxides | 5.0 |
| Gelling agent (DC Odessey ®) | 5.0 |
| Fragrance | 0.3 |

The manufacturing procedure is according to example 1.

### EXAMPLE 7

### Comparative Test

The lipstick of example 6 was tested with a group of 12 test persons. The lipstick was applied on the lips in usual manner by the test persons. After a drying time of 2 min. the lips were dabbed with a white cloth 3-4 times. After that the white clothes were visual examined by the test leader. No visual traces of the lipstick were found on the cloth in any case. The test persons stated at inquiry in any case an unusual high gloss of the lips after apply.

1. Decorative cosmetic product having a high water content, which comprises 70-92% by weight of water, 2.0-30% by weight of pigments, 2-8% by weight of a gel-forming agent with emulsifying properties comprising a mixture of sodium acrylate copolymers, hydrogenated polyisobutene, plant-based phospholipids, a polyglyceryl acylate and an oil,
and the remainder up to 100% of said product is made up of cosmetic auxiliaries, carriers, active agents or mixtures thereof, all percentages being relative to the product's total weight.

2. Product according to claim 1, wherein the product is free of waxes, silicone fluids, monovalent C₁₋₄ alcohols, paraffinic hydrocarbons or mixtures thereof.

3. Product according to claim 1, wherein the content of water is in the range of 73-90% by weight of water, particularly 84-90% by weight.

4. Product according to claim 1, wherein the content of pigments is in the range of 5-20 by weight, particularly 8-15% by weight.

5. Product according to claim 4, wherein the pigments are untreated pigments.

6. Product according to claim 1, wherein the content of the gelling agent is in the range of 3-7% by weight.

7. Product according to claim 1, wherein the sodium acrylate copolymer is a copolymer of sodium acrylate with acryloyldimethyl taurate.

8. Product according to claim 1, wherein the phospholipid is stemming from soya oil, sunflower seed oil, rice oil or mixtures thereof.

9. Product according to claim 1, wherein the polyglyceryl acylate is a polyglyceryl stearate, polyglyceryl distearate or polyglyceryl linoleate, particularly a polyglyceryl-10 stearate.

10. Product according to claim 1, wherein the product is a lipstick, concealer, eye shadow, make-up, liner, mascara, foundation, rouge, eye-liner or lip-liner.

11. Product according to claim 10, wherein the product is a transfer resistant lipstick.
